(19) United States
(12) Patent Application Publication
(10) Pub. No.: US 2011/0021886 A1
(43) Pub. Date: Jan. 27, 2011

(54) Device and Method for Reducing the Heat Loss of Infants

(76) Inventor: Cheryl Briggs, Annapolis, MD (US)

Correspondence Address: CAPITAL LEGAL GROUP, LLC, 1100 River Bay Road, Annapolis, MD, 21409, US

(21) Appl. No.: 12/841,109

(22) Filed: Jul. 21, 2010

Related U.S. Application Data
(60) Provisional application No. 61/227,490, filed on Jul. 22, 2009.
(60) Provisional application No. 61/291,235, filed on Dec. 30, 2009.

(51) Int. Cl. A61B 5/00

(52) U.S. Cl. 600/301

(57) ABSTRACT

A device and method for reducing heat loss of a infant is provided. In one embodiment, the device includes a head portion having a face aperture, a middle portion connected to said head portion and having a belly aperture, wherein said belly aperture is positioned substantially above an umbilical cord of an infant disposed in the device, said middle portion having a closeable opening extending from said face aperture to said belly aperture, and a lower portion connected to said middle portion and having a leg compartment.

### CROSS REFERENCE TO RELATED APPLICATIONS

This application claims priority to U.S. Provisional No. 61/227,490 filed Jul. 22, 2009 and to U.S. Provisional Appl. No. 60/268,519, and U.S. Provisional Appl. No. 61/291,235 filed Dec. 30, 2009, both of which are incorporated herein by reference in their entirety for all purposes.

### BACKGROUND OF THE INVENTION

The present invention generally relates to devices and methods for providing infant care and more particularly, to devices and methods for reducing heat loss of infants.

Hypothermia of a preterm infant is a challenge to the neonatal care provider. Despite advances in technology, studies have demonstrated an association between hypothermia and increased morbidity and mortality in very low birth-weight infants. Such very low birth-weight infants (VLBW infants) are at increased risk for hypothermia even when treated according to current recommendations to dry, remove wet linen, and place on a radiant warmer. Various techniques have been introduced to decrease hypothermia, with each designed to address a different method of heat loss.

Infants lose heat via four mechanisms that include radiant, convective, evaporative, and conductive.

Some embodiments of the present invention may provide a device and method for reducing the heat loss of infants and especially premature infants.

### BRIEF DESCRIPTION OF THE DRAWINGS

The invention is further described in the detailed description that follows, by reference to the noted drawings by way of non-limiting illustrative embodiments of the invention, in which like reference numerals represent similar parts throughout the drawings. As should be understood, however, the invention is not limited to the precise arrangements and instrumentalities shown. In the drawings:

FIG. 1 depicts device an infant insulation device according to an example embodiment of the present invention;

FIG. 2 depicts device an infant insulation device according to another example embodiment of the present invention; and

FIG. 3 depicts device an infant insulation device according to another example embodiment of the present invention.

### DETAILED DESCRIPTION OF ILLUSTRATIVE EMBODIMENTS

In the following description, for purposes of explanation and not limitation, specific details are set forth, such as particular materials, manufacturing mechanisms, sizes, etc. in order to provide a thorough understanding of the present invention.

However, it will be apparent to one skilled in the art that the present invention may be practiced in other embodiments that depart from these specific details. Detailed descriptions of well-known particular materials, manufacturing mechanisms, sizes, etc. are omitted so as not to obscure the description.

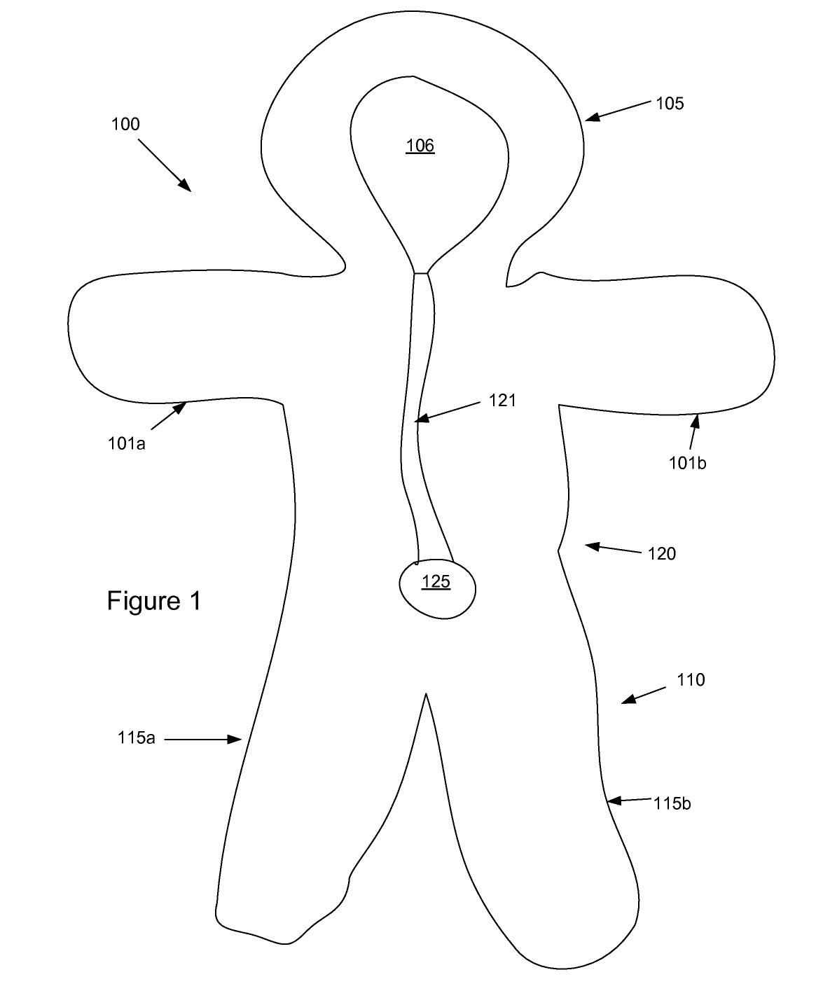

FIG. 1 depicts one example embodiment of the present invention. This example embodiment of the device 100 is formed of plastic or another non-permeable material and comprises head portion 105 in the shape of a hood and having an aperture 106. The aperture allows the care giver to see the face of the infant, to administer medicine, ventilation, etc. In some embodiments, the head portion may include a cotton lining. The lower portion 110 of the device includes first and second leg portions 115a and 115b for receiving the first and second legs of the infant. In some embodiments, one or both leg portions 115 may include a heel flap that allows the care giver access to the infant's heel to take blood. The heel flap may be held in the closed position via an adhesive, velcro or other suitable means. The middle portion 120 includes first and second arm portions 101a and 101b configured to receive the first and second arms of the infant.

In addition, the middle portion 120 includes a belly aperture 125 that is positioned approximate to where the infant's belly button or umbilical cord would be located. In some instances, the care giver will need access to the umbilical cord. Consequently, the belly aperture 125 allows access to the cord without the need to remove the infant from the device or to open the device. In some embodiments, a belly aperture flap (not shown) may be included to cover the belly aperture 125 but that allows easy access to the infant's cord. The belly aperture flap may be held in the closed position via an adhesive or other suitable means.

In addition, the middle portion 120 includes a reclosable opening 121. The opening 121 is preferably can be repeatedly fixedly closed and opened by the caregiver. The closing allows the heat loss to be reduced. The ability to open the opening 121 allows access to the torso of the infant and/or to remove the infant from the device. The opening 121 may be fixedly closed via any suitable means such as via Velcro, snaps, a zipper, buttons, an adhesive, etc.

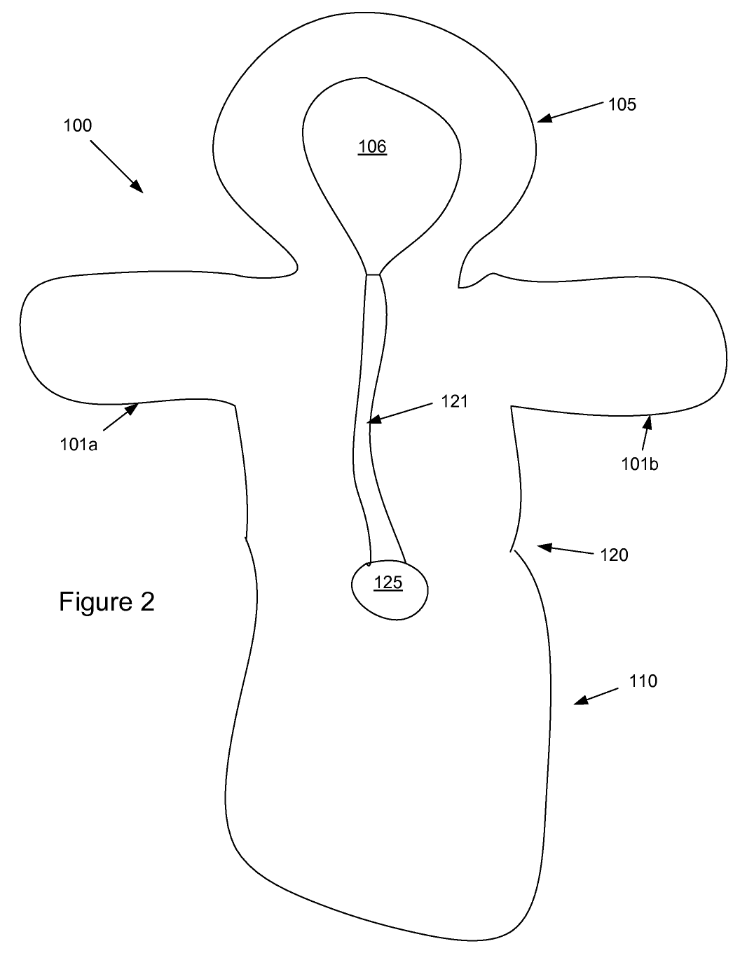

FIG. 2 depicts another example embodiment in which the lower portion 110 does not include separate leg portions 110 and instead includes a single compartment configured to receive the infants legs. Each leg portion 110 may include a closeable opening (and flap if necessary)

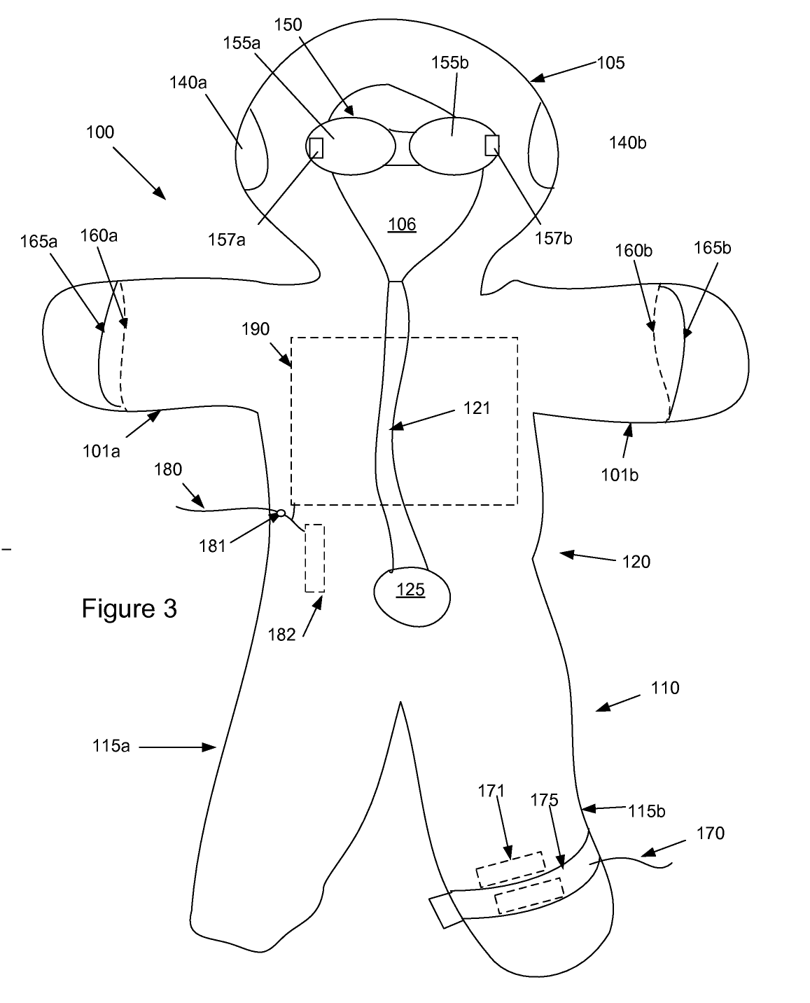

FIG. 3 depicts another example embodiment that is similar to the embodiment illustrated in FIG. 1 but further includes an eye covering mechanism 150 that includes a first 155a and second 155b eye cover. The eye covering mechanism 150 of this embodiment is removably attached to the head portion 105 via first 157a and second 157b eye cover attachments, which in this embodiment are velcro attachments, but in other embodiments may comprise snaps, buttons, an adhesive, or any other suitable attachment mechanism. The eye covering mechanism 150 thus prevents bright light from disturbing the infant. The eye covers 155 may be 100% opaque, semi-opaque, designed to filter certain wavelengths of light, or, in other embodiments may be semi-transparent or transparent (and therefore primarily act to protect the eyes from physical impact). Different eye covering mechanisms 150 (with different eye covers 155) may be attached to the head portion 105 at different times (e.g., different times of the day, night or during examinations or other procedures). Of course, the eye covering mechanism 155 may be removed at selected times (e.g., day time) and/or during certain procedures (e.g., examinations).

The embodiment of FIG. 3 also includes first and second ear pads 140a,b that may provide insulation against ambient noise. More specifically, the ear pads 140 may be designed to provide greater noise (audio) resistance (e.g., dampening, insulation, etc.) than other portions of the head portion 105 and may be removably attached to the head portion 105. In this embodiment, the head portion 105 includes a first and second aperture (not shown) located to allow access to each ear of the infant. Thus, upon removing the ear pads 140 the medical personnel can access the ears of the infants. In other embodiments, the ear pads 140 may each comprise a flap that is fixedly attached on one side of the ear aperture and is removably attached around the remaining circumference of the aperture (e.g., via velcro, a snap, an adhesive, a button, etc.). In yet another embodiment, the head portion does not include any apertures and the ear pads 140 simply reduce the noise experienced by the infant in which case the ear pads may not be removable.

Finally, the embodiment of FIG. 3 also may include appendage openings 160a,b to allow medical personnel to access to the infants arms to perform an arterial blood draw. In this embodiment, each opening 160 is covered with a flap 165 that may optionally be fastened over the opening with Velcro, a snap, an adhesive, a button or other suitable method. Similar openings (and flaps) may additionally or alternately be provided on the leg portions 115 to allow access to the leg or feet (e.g., the heal). The openings and flaps may provide IV (intravenous) access, endrotracheal tube access, and access of any other necessary medical devices.

In some other embodiments, the device may be attached to a warming mattress and therefore the device may include snaps, velcro or other fastening mechanisms to do so. In some instants, the infant in the device may be placed on a warming mattress and under a radiant warmer. In still other embodiment, a heating element 190 may be formed in a layer of the device and (1) have a battery power supply; and/or (2) connect to an external power supply. Preferably the heating element has an adjustable heating feature and a timer (for timing the on and off) and is also responsive to the measured temperature so that the heating elements maintains the temperature within a predetermine temperature range (e.g., by increasing and decreasing the heat produced). Such a heating element may be used when the infant is removed from the incubator. The heating element 190 may be integrated into the entire device 100 or into only one or more portions (e.g., the middle portion 120 and/or head portion 105). The entire device may be lined with a liner of a suitable material that does not stick to the infant's skin such as cotton. Alternately, only the head portion 105 may have a liner and the body portion does not.

In addition, a temperature sensor 182 may also be attached to or integrated into the insulator device 100 on the inner most lining (e.g., next to the infant's skin) that provide a digital reading of the temperature through a wire such as wire 180 in FIG. 3 (or wireless) to an external display so that care givers can know the temperature of the baby. The temperature sensor 182 may also provide data to the heating element. Alternately, a thermometer may be taped to the underarm (or located at another desired location) and supply temperature data to the display (e.g., wirelessly or via wire). If a wire 180 is used, the device 100 may include an aperture 181 for the wire or, alternately, the wire may be integrated into the device 100 during manufacturing so that the wire extends from inside the device 100 to the outside through an aperture 181. Some embodiments may be formed of “semi-permeable” material which allows moisture to travel external from the infant but prevents moisture from traveling the reverse direction.

In addition, the insulating device 100 may include an oxygen sensor 171 to measure the oxygen content (O₂) of the blood of the infant. As illustrated in FIG. 3, the oxygen sensor may be integrated into a appendage such as an arm portion 1101 (e.g., near the wrist) or leg portion 115 (e.g., near the ankle) and include a fastening mechanism 175 that allows the leg portion (or arm portion) to be tightened around the appendage so as to ensure that the oxygen sensor 171 (e.g., having a first and second portion that cooperate together) integrated into the leg portion comes into contact with the infant's skin so that the oxygen sensor 171 can accurately and reliably measure the oxygen content. In this example embodiment, the fastening mechanism comprises a velcro strap that is fixedly attached to the leg portion 115b (e.g., via an adhesive or sewn in), but other embodiments may use other fastening means such as multiple buttons, snaps, a buckle, etc. A wire 170 may extend from the sensor to the appropriate equipment for displaying the oxygen content. In other embodiments, the oxygen sensor may have a battery and transmit the data wirelessly to the equipment.

Thus, some of the embodiments of the present invention may comprise a head portion having a face aperture; a middle portion connected to the head portion and having a belly aperture; wherein the belly aperture is positioned substantially above an umbilical cord of an infant disposed in the device; the middle portion having a closeable opening extending from the face aperture to said belly aperture; a lower portion connected to said middle portion; an eye covering mechanism removably attached to the head portion to at least partially cover the face aperture to thereby cover the eyes of the infant; a temperature sensor attached into the device to assess a temperature inside the device; and an oxygen sensor attached into the device to assess a blood oxygen content of an infant in the device; and a fastening mechanism to urge one or both portions of the oxygen sensor into contact with the skin of the infant.

It is to be understood that the foregoing illustrative embodiments have been provided merely for the purpose of explanation and are in no way to be construed as limiting of the invention. Words used herein are words of description and illustration, rather than words of limitation. In addition, the advantages and objectives described herein may not be realized by each and every embodiment practicing the present invention. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention.

What is claimed is:

1. A device for reducing heat loss of an infant, comprising:
a head portion having a face aperture;
a middle portion connected to said head portion and having a belly aperture;
wherein said belly aperture is positioned substantially above an umbilical cord of an infant disposed in the device;
said middle portion having a closeable opening extending from said face aperture to said belly aperture;
a lower portion connected to said middle portion;
an eye covering mechanism removably attached to the head portion to at least partially cover the face aperture to thereby cover the eyes of the infant;
a temperature sensor attached into the device to assess a temperature inside the device; and
an oxygen sensor attached into the device to assess a blood oxygen content of an infant in the device.

2. A device for reducing heat loss of an infant, comprising:
a head portion having a face aperture;
a middle portion connected to said head portion and having a belly aperture;
wherein said belly aperture is positioned substantially above an umbilical cord of an infant disposed in the device;
said middle portion having a closeable opening extending from said face aperture to said belly aperture; and
a lower portion connected to said middle portion and having a leg compartment.

3. The device of claim 2, wherein said head portion includes a liner.

4. The device of claim 2, wherein said belly aperture includes a closeable flap.

5. The device of claim 2, further comprising an oxygen sensor integrated into the lower portion.

6. The device of claim 2, further comprising an eye covering mechanism removably attached to the head portion to partially cover the face aperture to thereby cover the eyes of the infant.

7. The device of claim 2, further comprising a first portion and a second portion connected to said middle portion and each configured to receive an arm of the infant; and
wherein at least one of said first and second arm portions includes an opening and a flap covering said opening.

8. The device of claim 2, further comprising a first and second ear pad removably attached to said head portion and each located to cover an ear of the infant.

9. The device of claim 2, further comprising a temperature sensor for assessing the temperature inside the device and configured to provide temperature data to a display external to the display.

10. The device of claim 2, further comprising a heating element integrated into at least said middle portion.

11. The device of claim 2, wherein said lower portion includes a first leg portion and a second leg portion.

12. A method of forming a device, comprising:
providing a head portion having a face aperture;
providing a middle portion connected to said head portion and having a belly aperture;
wherein said belly aperture is positioned to substantially above an umbilical cord of an infant disposed in the device;
said middle portion having a closeable opening extending from said face aperture to said belly aperture and configured to be fixed closed; and
providing a lower portion connected to said middle portion and having a leg compartment.
placing an infant in the device;
closing the opening of the device;
connecting at least one medical item to the infant.

13. The method according to claim 9, further comprising placing the infant in the device under a radiant warmer.

14. The method according to claim 12, further comprising accessing the umbilical cord of the infant through the belly aperture of the device.

15. The method according to claim 12, wherein said device further comprises an eye covering mechanism removably attached to the head portion to partially cover the face aperture to thereby cover the eyes of the infant.

16. The method according to claim 12, wherein said device further comprises a first arm compartment and a second arm compartment connected to said middle portion and each configured to receive an arm of the infant;
wherein said first and second arm compartment each include an opening and a flap covering said opening.

17. The method according to claim 12, wherein said device further comprises a first and second ear pad removably attached to said head portion and each located to cover an ear of the infant.

18. The method according to claim 12, further comprising placing the infant in the device on a warming mattress.

19. The method according to claim 12, wherein the at least one medical item is selected from the group of: an intravenous medication and an endrotracheal tube.

20. The method according to claim 12, providing a temperature sensor integrated into the device.
